SEQUENCE LISTING (2 sequences)

SEQ ID NO: 1 - DNA, 856 bp, Lawsonia intracellularis
  CDS (80)..(823)
atggctataagcgattgaataacagaaaataacacctatgcctgaaattttcgacgcgtcgaaatttttagaggaaaccatgaaaaaactactccttttgttatctattctgtttctaaccccaagtattaccttggcggaaggtaatactttcaatgatagtttcaacaaggctaagcgcatactgcaagatgaggtgtattacgaccaccaagttacactatactgcggatatgaatatgatgaccaaaaaaggatatgtctccctgatggatttatagcagagaaacatcaaaaaagatcatataaaattgagtgggaacatagtgtgcctgctgagaattttggcagagcttttactgaatggcgcgaaggtcatcctctttgtgtagataataaaggtaaaagtttcaaaggacgaaaatgtgcagaaaaagtaaataaaacatatagatatatgcagtctgatatgtacaatttgtttccagcagtcgggtctgtcaatgctgcgagaagcaataagcaatactcagagttacttggagttcaatctgcttttggaacgtgtgaggcaaaaatagatgggaatagattcgaaccaccggatagagctaaaggtcaagtagcccgtgctgctctttatatggataaagagtacaaggaatacaatctaagtcgtcagcaaagaagactttttgaggcttggagtaatatgtatccagtcgatgaatgggagtgtacacgagccaaacgaatcgaatctatacagggaaatgaaaatatttttgtaaaaaatatgtgtatcgaaaaggggttatggtaaacaaacgaggacaatataaatactacctaagta

SEQ ID NO: 2 - protein, 247 aa, Lawsonia intracellularis
MKKLLLLLSILFLTPSITLAEGNTFNDSFNKAKRILQDEVYYDHQVTLYCGYEYDDQKRICLPDGFIAEKHQKRSYKIEWEHSVPAENFGRAFTEWREGHPLCVDNKGKSFKGRKCAEKVNKTYRYMQSDMYNLFPAVGSVNAARSNKQYSELLGVQSAFGTCEAKIDGNRFEPPDRAKGQVARAALYMDKEYKEYNLSRQQRRLFEAWSNMYPVDEWECTRAKRIESIQGNENIFVKNMCIEKGLW

(19) United States
(12) Patent Application Publication
(10) Pub. No.: US 2007/0212373 A1
(43) Pub. Date: Sep. 13, 2007

(54) Lawsonia Intracellularis 26 Kd Subunit Vaccine

(75) Inventor: Paul Vermeij, St. Anthonis (NL)

Correspondence Address: INTERVET INC.;PATENT DEPARTMENT, PO BOX 318, MILLSBORO, DE, 19966-0318, US

(73) Assignee: Akzo Nobel N.V., Arnhem (NL)

(21) Appl. No.: 10/580,709

(22) PCT Filed: Dec. 8, 2004
(86) PCT No.: PCT/EP04/53342

§ 371 Date: May 25, 2006

(51) Int. Cl. A61K 48/00; C07H 21/04; A61K 39/02; C07K 14/195; C12N 1/21

(52) U.S. Cl. 424/200.1; 514/44; 435/252.3; 536/23.7; 530/350

(57) ABSTRACT

The present invention relates i.a. to nucleic acids encoding novel Lawsonia intracellularis proteins. It furthermore relates to DNA fragments, recombinant DNA molecules and live recombinant carriers comprising these sequences. Also it relates to host cells comprising such nucleic acids, DNA fragments, recombinant DNA molecules and live recombinant carriers. Moreover, the invention relates to proteins encoded by these nucleotide sequences and to their use for the manufacturing of vaccines. The invention also relates to vaccines for combating Lawsonia intracellularis infections and methods for the preparation thereof.

Finally the invention relates to diagnostic tests for the detection of Lawsonia intracellularis antigens and of antibodies against Lawsonia intracellularis.

The present invention relates i.a. to nucleic acids encoding novel Lawsonia intracellularis proteins, to DNA fragments, recombinant DNA molecules and live recombinant carriers comprising these sequences, to host cells comprising such nucleic acids, DNA fragments, recombinant DNA molecules and live recombinant carriers, to proteins encoded by these nucleotide sequences and to their use for the manufacturing of vaccines, to vaccines for combating Lawsonia intracellularis infections and methods for the preparation thereof and to diagnostic tests for the detection of Lawsonia intracellularis antigens and for the detection of antibodies against Lawsonia intracellularis.

Porcine proliferative enteropathy (PPE or PE) has become an important disease of the modern pig industry world-wide. The disease affects 15% to 50% of the growing herds and up to 30% of the individual animals in established problem herds. Today annual economical losses have been estimated US$ 5-10 in extra feed and facility time costs per affected pig. PPE is a group of chronic and acute conditions of widely differing clinical signs (death, pale and anaemic animals, watery, dark or bright red diarrhea, depression, reduced appetite and reluctance to move, retarded growth and increased FCR). However there are two consistent features. The first, a pathological change only visible at necropsy, is a thickening of the small intestine and colon mucosa. The second is the occurrence of intracytoplasmatic small-curved bacteria in the enterocytes of the affected intestine. These bacteria have now been established as the etiological agent of PPE and have been name Lawsonia intracellularis.

Over the years Lawsonia intracellularis has been found to affect a large group of animals including monkeys, rabbits, ferrets, hamsters, fox, horses, and other animals as diverse as ostrich and emoe. Lawsonia intracellularis is a gram-negative, flagellated bacterium that multiplies in eukryotic enterocytes only and no cell-free culture has been described. In order to persist and multiply in the cell Lawsonia intracellularis must penetrate dividing crypt cells. The bacterium associates with the cell membrane and quickly enters the enterocyte via an entry vacuole. This then rapidly breaks down (within 3 hours) and the bacteria flourish and multiply freely in the cytoplasm. The mechanisms by which the bacteria cause infected cells to fail to mature, continue to undergo mitosis and form hypoplastic crypt cells is not yet understood.

The current understanding of Lawsonia intracellularis infection, treatment and control of the disease has been hampered by the fact that Lawsonia intracellularis can not be cultivated in cell-free media. Although there are reports of successful co-culturing Lawsonia intracellularis in rat enterocytes this has not lead to the development of inactivated vaccines for combating Lawsonia intracellularis, although there clearly is a need for such vaccines.

It is an objective of the present invention to provide a vaccine for combating Lawsonia intracellularis infection.

It was surprisingly found now, that Lawsonia intracellularis is produces a novel protein that is capable of inducing protective immunity against Lawsonia intracellularis.

The novel protein will be referred to as the 26 kD protein.

The amino acid sequence of the novel protein is presented in sequence identifier SEQ ID NO: 2. The gene encoding this protein has been sequenced and its nucleic acid sequence is shown in sequence identifier SEQ ID NO: 1. The gene will also be referred to in the Examples as “gene 5608”.

It is well-known in the art, that many different nucleic acid sequences can encode one and the same protein. This phenomenon is commonly known as wobble in the second and especially the third base of each triplet encoding an amino acid. This phenomenon can result in a heterology of about 30% for two nucleic acid sequences still encoding the same protein. Therefore, two nucleic acid sequences having a sequence homology of about 70% can still encode one and the same protein.

Thus, one embodiment relates to nucleic acids encoding a Lawsonia intracellularis protein and to parts of that nucleic acid that encode an immunogenic fragment of that protein, wherein those nucleic acids or parts thereof have a level of homology with the nucleic acid of which the sequence is given in SEQ ID NO: 1 of at least 90%.

Preferably, the nucleic acid encoding this Lawsonia intracellularis protein or the part of said nucleic acid has at least 92%, preferably 94%, more preferably 95% and even more preferably 96% homology with the nucleic acid having the sequence given in SEQ ID NO: 1. Even more preferred is a homology level of 98% or even 100%.

The level of nucleotide homology can be determined with the computer program “BLAST 2 SEQUENCES” by selecting sub-program: “BLASTN” that can be found at www.ncbi.nln.nih.gov/blast/b12seq/b12.html.

A reference for this program is Tatiana A. Tatusova, Thomas L. Madden FEMS Microbiol. Letters 174: 247-250 (1999). Parameters used are the default parameters: Reward for a match:+1. Penalty for a mismatch: −2. Open gap: 5. Extension gap: 2. Gap x_dropoff: 50.

Another approach for deciding if a certain nucleic acid is or is not a nucleic acid according to the invention relates to the question if that certain nucleic acid does hybridise under stringent conditions to nucleic acids having the nucleotide sequence as depicted in SEQ ID NO: 1.

If a nucleic acid hybridises under stringent conditions to the nucleotide sequence as depicted in SEQ ID NO: 1, it is considered to be a nucleic acid according to the invention.

The definition of stringent conditions follows from the formula of Meinkoth and Wahl (1984. Hybridization of nucleic acids immobilized on solid supports. Anal. Biochem. 138:267-284.)
Tm=[81.5° C.+16.6(log M)+0.41(%GC)−0.61(%formamide)−500/L]−1° C./1% mismatch

In this formula, M is molarity of monovalent cations; % GC is the percentage of guanosine and cytosine nucleotides in the DNA; L is the length of the hybrid in base pairs.

Stringent conditions are those conditions under which nucleic acids or fragments thereof still hybridise, if they have a mismatch of 10% at the most, to the nucleic acid having the sequence depicted in SEQ ID NO: 1.

Since the present invention discloses nucleic acids encoding novel Lawsonia intracellularis proteins, it is now for the first time possible to obtain these proteins in sufficient quantities. This can e.g. be done by using expression systems to express the genes encoding the proteins.

Therefore, in a more preferred embodiment, the invention relates to DNA fragments comprising a nucleic acid according to the invention. Such DNA fragments can e.g. be plasmids, into which a nucleic acid according to the invention is cloned. Such DNA fragments are e.g. useful for enhancing the amount of DNA for use as a primer, as described below.

An essential requirement for the expression of the nucleic acid is an adequate promoter functionally linked to the nucleic acid, so that the nucleic acid is under the control of the promoter. It is obvious to those skilled in the art that the choice of a promoter extends to any eukaryotic, prokaryotic or viral promoter capable of directing gene transcription in cells used as host cells for protein expression.

Therefore, an even more preferred form of this embodiment relates to a recombinant DNA molecule comprising a DNA fragment or a nucleic acid according to the invention that is placed under the control of a functionally linked promoter. This can be accomplished by means of e.g. standard molecular biology techniques. (Sambrook, J. and Russell, D. W., Molecular cloning: a laboratory manual, 2001. ISBN 0-87969-577-3). Functionally linked promoters we promoters that are capable of controlling the transcription of the nucleic acids to which they are linked.

Such a promoter can be a Lawsonia promoter e.g. the promoter involved in in vivo expression of the gene encoding the 26 kD gene, provided that that promoter is functional in the cell used for expression. It can also be a heterologous promoter. When the host cells are bacteria, useful expression control sequences which may be used include the Tip promoter and operator (Goeddel, et al., Nucl. Acids Res., 8, 4057, 1980); the lac promoter and operator (Chang, et al., Nature, 275, 615, 1978); the outer membrane protein promoter (Nakamura, K. and Inouge, M., EMBO J., 1, 771-775, 1982); the bacteriophage lambda promoters and operators (Remaut, E. et al., Nucl. Acids Res., 11, 4677-4688, 1983); the α-amylase (B. subtilis) promoter and operator, termination sequences and other expression enhancement and control sequences compatible with the selected host cell.

When the host cell is yeast, useful expression control sequences include, e.g., α-mating factor. For insect cells the polyhedrin or p10 promoters of baculoviruses can be used (Smith, G. E. et al., Mol. Cell. Biol. 3, 2156-65, 1983). When the host cell is of mammalian origin illustrative useful expression control sequences include the SV-40 promoter (Berman, P. W. et al., Science, 222, 524-527, 1983) or the metallothionein promoter (Brinster, R. L., Nature, 296, 3942, 1982) or a heat shock promoter (Voellmy et al., Proc. Natl. Acad. Sci. USA, 82, 4949-53, 1985).

Bacterial, yeast, fungal, insect and mammalian cell expression systems are very frequently used systems. Such systems are well-known in the art and generally available, e.g. commercially through Invitrogen (www.invitrogen.com), Novagen (www.merckbiosciences.de) or Clontech Laboratories, Inc. 4030 Fabian Way, Palo Alto, Calif. 94303-4607, USA. Next to these expression systems, parasite-based expression systems are very attractive expression systems. Such systems are e.g. described in the French Patent Application with Publication number 2 714 074, and in US, NTIS Publication No U.S. Ser. No. 08/043,109 (Hoffman, S. and Rogers, W.: Public. Date 1 Dec. 1993).

A still even more preferred form of this embodiment of the invention relates to Live Recombinant Carriers (LRCs) comprising a nucleic acid encoding the 26 kD protein or an immunogenic fragment thereof according to the invention, a DNA fragment according to the invention or a recombinant DNA molecule according to the invention. Such carriers are e.g. bacteria and viruses. These LRCs are micro-organisms or viruses in which additional genetic information, in this case a nucleic acid encoding the 26 kD protein or an immunogenic fragment thereof according to the invention has been cloned.

Animals infected with such LRCs will produce an immunogenic response not only against the immunogens of the carrier, but also against the immunogenic parts of the protein(s) for which the genetic code is additionally cloned into the IRC, e.g. the 26 kD protein.

As an example of bacterial LRCs, attenuated Salmonella stains known in the art can attractively be used.

Live recombinant carrier parasites have i.a been described by Vermeulen, A. N. (Int Journ. Parasitol. 28: 1121-1130 (1998))

Also, LRC viruses may be used as a way of transporting the nucleic acid into a target cell. Live recombinant carrier viruses are also called vector viruses. Viruses often used as vectors are Vaccinia viruses (Panicali et al; Proc. Natl. Acad. Sci. USA, 79: 4927 (1982), Herpesviruses (E.P.A. 0473210A2), and Retroviruses (Valerio, D. et al.; in Baum, S. J., Dicke, K. A., Lotzova, E. and Pluznik, D. H. (Eds.), Experimental Haematology today-1988. Springer Verlag, New York: pp. 92-99 (1989)).

The technique of in vivo homologous recombination, well-known in the art, can be used to introduce a recombinant nucleic acid into the genome of a bacterium, parasite or virus of choice, capable of inducing expression of the inserted nucleic acid according to the invention in the host animal.

Finally another form of this embodiment of the invention relates to a host cell comprising a nucleic acid encoding a protein according to the invention, a DNA fragment comprising such a nucleic acid or a recombinant DNA molecule comprising such a nucleic acid under the control of a functionally linked promoter. This form also relates to a host cell containing a live recombinant carrier containing a nucleic acid molecule encoding a 26 kD protein or a fragment thereof according to the invention.

A host cell may be a cell of bacterial origin, e.g. Escherichia coli, Bacillus subtilis and Lactobacillus species, in combination with bacteria-based plasmids as pBR322, or bacterial expression vectors as pGEX, or with bacteriophages. The host cell may also be of eukaryotic origin, e.g. yeast-cells in combination with yeast-specific vector molecules, or higher eukaryotic cells like insect cells (Luckow et al.; Bio-technology 6: 47-55 (1988)) in combination with vectors or recombinant baculoviruses, plant cells in combination with e.g. Ti-plasmid based vectors or plant viral vectors (Barton, K. A. et al.; Cell 32: 1033 (1983), mammalian cells like Hela cells, Chinese Hamster Ovary cells (CHO) or Crandell Feline Kidney-cells, also with appropriate vectors or recombinant viruses.

Another embodiment of the invention relates to the novel proteins and to immunogenic fragments thereof according to the invention.

The concept of immunogenic fragments will be defined below. One form of this embodiment relates i.a. to Lawsonia intracellularis proteins that have an amino acid sequence that is at least 90% homologous to the amino acid sequence as depicted in SEQ ID NO: 2 and to immunogenic fragments of said protein.

In a preferred form, the embodiment relates to such Lawsonia intracellularis proteins that have a sequence homology of at least 92%, preferably 94%, more preferably 96% homology to the amino acid sequence as depicted in SEQ ID NO: 2 and to immunogenic fragments of such proteins.

Even more preferred is a homology level of 98% or even 100%. The level of protein homology can be determined with the computer program “BLAST 2

### SEQUENCES” by selecting sub-program: “BLASTP”, that can be found at www.ncbi.nlm.nih.gov/blast/bl2seq/b12.html.

A reference for this program is Tatiana A. Tatusova, Thomas L. Madden FEMS Microbiol. Letters 174: 247-250 (1999). Matrix used: “blosum62”. Parameters used are the default parameters:

Open gap: 11. Extension gap: 1. Gap x_dropoff: 50.

It will be understood that, for the particular proteins embraced herein, natural variations can exist between individual Lawsonia intracellularis strains. These variations may be demonstrated by (an) amino acid difference(s) in the overall sequence or by deletions, substitutions, insertions, inversions or additions of (an) amino acid(s) in said sequence. Amino acid substitutions which do not essentially alter biological and immunological activities, have been described, e.g. by Neurath et al. in “The Proteins” Academic Press New York (1979). Amino acid replacements between related amino acids or replacements which have occurred frequently in evolution are, inter alia, Ser/Ala, Ser/Gly, Asp/Gly, Asp/Asn, Ile/Val (see Dayhof, M. D., Atlas of protein sequence and structure, Nat. Biomed. Res. Found., Washington D.C., 1978, vol. 5, suppl. 3). Other amino acid substitutions include Asp/Glu, Thr/Ser, Ala/Gly, Ala/Thr, Ser/Asn, Ala/Val, Thr/Phe, Ala/Pro, Lys/Arg, Leu/Ile, Leu/Val and Ala/Glu. Based on this information, Lipman and Pearson developed a method for rapid and sensitive protein comparison (Science, 227, 1435-1441, 1985) and determining the functional similarity between homologous proteins. Such amino acid substitutions of the exemplary embodiments of this invention, as well as variations having deletions and/or insertions are within the scope of the invention as long as the resulting proteins retain their immune reactivity. This explains why Lawsonia intracellularis proteins according to the invention, when isolated from different field isolates, may have homology levels of about 90%, while still representing the same protein with the same immunological characteristics. Those variations in the amino acid sequence of a certain protein according to the invention that still provide a protein capable of inducing an immune response against infection with Lawsonia intracellularis or at least against the clinical manifestations of the infection are considered as “not essentially influencing the immunogenicity”.

When a protein is used for e.g. vaccination purposes or for raising antibodies, it is however not necessary to use the whole protein. It is also possible to use a fragment of that protein that is capable, as such or coupled to a carrier such as e.g. KLH, of inducing an immune response against that protein, a so-called immunogenic fragment. An “immunogenic fragment” is understood to be a fragment of the full-length protein that still has retained its capability to induce an immune response in the host, i.e. comprises a B- or T-cell epitope. At this moment, a variety of techniques is available to easily identify DNA fragments encoding antigenic fragments (determinants). The method described by Geysen et al. (Patent Application WO 84/03564, Patent Application WO 86/06487, U.S. Pat. No. 4,833,092, Proc. Natl Acad. Sci. 81: 3998-4002 (1984), J. Imm. Meth. 102, 259-274 (1987), the so-called PEPSCAN method is an easy to perform, quick and well-established method for the detection of epitopes; the immunologically important regions of the protein. The method is used world-wide and as such well-known to man skilled in the art. This (empirical) method is especially suitable for the detection of B-cell epitopes. Also, given the sequence of the gene encoding any protein, computer algorithms are able to designate specific protein fragments as the immunologically important epitopes on the basis of their sequential and/or structural agreement with epitopes that are now known. The determination of these regions is based on a combination of the hydrophilicity criteria according to Hopp and Woods (Proc. Natl. Acad. Sci. 78: 38248-3828 (1981)), and the secondary structure aspects according to Chou and Fasman (Advances in Enzymology 47: 45-148 (1987) and U.S. Pat. No. 4,554,101). T-cell epitopes can likewise be predicted from the sequence by computer with the aid of Berzofsky's amphiphilicity criterion (Science 235, 1059-1062 (1987) and U.S. Patent application NTIS U.S. Ser. No. 07/005,885). A condensed overview is found in: Shan Lu on common principles: Tibtech 9: 238-242 (1991), Good et al. on Malaria epitopes; Science 235: 1059-1062 (1987), Lu for a review, Vaccine 10: 3-7 (1992), Berzowsky for HIV-epitopes; The FASEB Journal 5:2412-2418 (1991).

Therefore, one form of still another embodiment of the invention relates to vaccines capable of protecting pigs against Lawsonia intracellularis infection, that comprise a protein or an immunogenic fragment thereof, according to the invention as described above together with a pharmaceutically acceptable carrier.

Still another embodiment of the present invention relates to the proteins according to the invention for use in a vaccine.

Still another embodiment relates to the use of a protein according to the invention for the manufacturing of a vaccine for combating Lawsonia intracellularis infections.

One way of making a vaccine according to the invention is by biochemical purification of the proteins or immunogenic fragments thereof according to the invention from bacteria obtained through mucosal scrapings taken from the infected intestine wall. This is however a very time-consuming way of linking the vaccine.

It is therefore much more convenient to use the expression products of the genes encoding the proteins or immunogenic fragments thereof according to the invention in vaccines. The nucleic acid of the gene encoding the 26 kD protein is provided by the present invention.

Such vaccines based upon the expression products of these genes can easily be made by admixing a protein according to the invention or an immunogenic fragment thereof according to the invention with a pharmaceutically acceptable carrier as described below.

Alternatively, a vaccine according to the invention can comprise live recombinant carriers as described above, capable of expressing the proteins according to the invention or immunogenic fragments thereof according to the invention. Such vaccines, e.g. based upon a Salmonella carrier or a viral carrier infecting the enteric epithelium, or e.g. the respiratory epithelium have the advantage over subunit vaccines that they better mimic the natural way of infection of Lawsonia intracellularis. Moreover, their self-propagation is an advantage since only low amounts of the recombinant carrier are necessary for immunization.

Vaccines described above all contribute to active vaccination, i.e. the host's immune system is triggered by a protein according to the invention or an immunogenic fragment thereof, to make antibodies against these proteins.

Alternatively, such antibodies can be raised in e.g. rabbits or can be obtained from antibody-producing cell lines as described below. Such antibodies can then be administered to the host animal. This method of vaccination, passive vaccination, is the vaccination of choice when an animal is already infected, and there is no time to allow the natural immune response to be triggered. It is also the preferred method for vaccinating immune-compromised animals. Administered antibodies against Lawsonia intracellularis can in these cases bind directly to the bacteria This has the advantage that it immediately decreases or stops Lawsonia intracellularis growth.

Therefore, one other form of this embodiment of the invention relates to vaccines comprising antibodies against the 26 kD Lawsonia intracellularis proteins according to the invention.

Vaccines can also be based upon host cells as described above, that comprise the proteins or immunogenic fragments thereof according to the invention.

An alternative and efficient way of vaccination is direct vaccination with DNA encoding the relevant antigen. Direct vaccination with DNA encoding proteins has been successful for many different proteins. (As reviewed in e.g. Donnelly et al., The Immunologist 2: 20-26 (1993)).

This way of vaccination is very attractive for the vaccination of pigs against Lawsonia intracellularis infection.

Therefore, still other forms of this embodiment of the invention relate to vaccines comprising nucleic acids encoding a protein according to the invention or immunogenic fragments thereof according to the invention, and to vaccines comprising DNA fragments that comprise such nucleic acids.

Still other forms of this embodiment relate to vaccines comprising recombinant DNA molecules according to the invention.

DNA vaccines can easily be administered through intradermal application e.g. using a needle-less injector. This way of administration delivers the DNA directly into the cells of the animal to be vaccinated Amounts of DNA in the microgram range between 1 and 100 μg provide very good results.

In a further embodiment, the vaccine according to the present invention additionally comprises one or more antigens derived from other pig pathogenic organisms and viruses, or genetic information encoding such antigens.

Such organisms and viruses are preferably selected from the group of Pseudorabies virus, Porcine influenza virus, Porcine parvo virus, Transmissible gastro-enteritis virus, Rotavirus, Escherichia coli, Erysipelothrix rhusiopathiae, Bordetella bronchiseptica, Salmonella cholerasuis, Haemophilus parasuis, Pasteurella multocida, Stretococcus suis, Mycoplasma hyopneumoniae, Brachyspira hyodysentenae and Actinobacillus pleuropneumoniae.

All vaccines according to the present invention comprise a pharmaceutically acceptable carrier. A pharmaceutically acceptable carrier can be e.g. sterile water or a sterile physiological salt solution. In a more complex form the carrier can e.g. be a buffer.

Methods for the preparation of a vaccine comprise the admixing of a protein according to the invention, or an immunogenic fragment thereof and a pharmaceutically acceptable carrier.

Vaccines according to the present invention may in a preferred presentation also contain an adjuvant Adjuvants in general comprise substances that boost the immune response of the host in a non-specific manner. A number of different adjuvants are known in the art Examples of adjuvants are Freunds Complete and Incomplete adjuvant, vitamin E, non-ionic block polymers, muramyldipeptides, Quill A®, mineral oil e.g. Bayol® or Markol®, vegetable oil, and Carbopol® (a homopolymer), or Diluvac® Forte. The vaccine may also comprise a so-called “vehicle”. A vehicle is a compound to which the polypeptide adheres, without being covalently bound to it Often used vehicle compounds are e.g. aluminium hydroxide, -phosphate or -oxide, silica, Kaolin, and Bentonite.

A special form of such a vehicle, in which the antigen is partially embedded in the vehicle, is the so-called ISCOM (EP 109.942, EP 180.564, EP 242.380)

In addition, the vaccine may comprise one or more suitable surface-active compounds or emulsifiers, e.g. Span or Tween.

Often, the vaccine is mixed with stabilisers, e.g. to protect degradation-prone polypeptides from being degraded, to enhance the shelf-life of the vaccine, or to improve freeze-drying efficiency. Useful stabilisers are i.a SPGA (Bovarnik: et al.; J. Bacteriology 59: 509 (1950)), carbohydrates e.g. sorbitol, mannitol, trehalose, starch, sucrose, dextran or glucose, proteins such as albumin or casein or degradation products thereof, and buffers, such as alkali metal phosphates.

In addition, the vaccine may be suspended in a physiologically acceptable diluent It goes without saying, that other ways of adjuvating, adding vehicle compounds or diluents, emulsifying or stabilising a polypeptide are also embodied in the present invention.

Vaccines according to the invention can very suitably be administered in amounts ranging between 1 and 100 micrograms, although smaller doses can in principle be used. A dose exceeding 100 micrograms will, although immunologically very suitable, be less attractive for commercial reasons.

Vaccines based upon live attenuated recombinant carriers, such as the LRC-viruses and bacteria described above can be administered in much lower doses, because they multiply themselves during the infection. Therefore, very suitable amounts would range between 10³ and 10⁹ CFU/PFU for respectively bacteria and viruses.

Many ways of administration can be applied. Oral application is a very attractive way of administration, because the infection is an infection of the digestive tract A preferred way of oral administration is the packaging of the vaccine in capsules, known and frequently used in the art, that only disintegrate after they have passed the highly acidic environment of the stomach. Also, the vaccine could be mixed with compounds known in the art for temporarily enhancing the pH of the stomach.

Systemic application is also suitable, e.g. by intramuscular application of the vaccine. If this route is followed, standard procedures known in the art for systemic application are well-suited.

From a point of view of protection against disease, a quick and correct diagnosis of Lawsonia intracellularis infection is important.

Therefore it is another objective of this invention to provide diagnostic tools suitable for the detection of Lawsonia intracellularis infection.

A diagnostic test for the detection of Lawsonia intracellularis antibodies in sera can be e.g. a simple standard sandwich-ELISA-test in which 26 kD protein or antigenic fragments thereof according to the invention are coated to the wall of the wells of an ELISA-plate. A method for the detection of such antibodies is e.g. incubation of 26 kD protein or antigenic fragments thereof with serum from mammals to be tested, followed by e.g. incubation with a labelled antibody against the relevant mammalian antibody. A colour reaction can then reveal the presence or absence of antibodies against Lawsonia intracellularis. Another example of a diagnostic test system is e.g. the incubation of a Western blot comprising the 26 kD protein or an antigenic fragment thereof according to the invention, with serum of mammals to be tested, followed by analysis of the blot.

Thus, another embodiment of the present invention relates to diagnostic tests for the detection of antibodies against Lawsonia intracellulars. Such tests comprise a protein or a fragment thereof according to the invention.

A diagnostic test based upon the detection of antigenic material of the specific 26 kD protein of Lawsonia intracellularis antigens and therefore suitable for the detection of Lawsonia intracellularis infection can e.g. also be a standard ELISA test. In one example of such a test the walls of the wells of an ELISA plate are coated with antibodies directed against the 26 kD protein. After incubation with the material to be tested, labelled anti-Lawsonia intracellularis antibodies are added to the wells. A colour reaction then reveals the presence of antigenic material from Lawsonia intracellularis.

Therefore, still another embodiment of the present invention relates to diagnostic tests for the detection of antigenic material of Lawsonia intracellularis. Such tests comprise antibodies against a protein or a fragment thereof according to the invention.

The polypeptides or immunogenic fragments thereof according to the invention expressed as characterised above can be used to produce antibodies, which may be polyclonal, monospecific or monoclonal (or derivatives thereof). If polyclonal antibodies are desired, techniques for producing and processing polyclonal sera are well-known in the art (e.g. Mayer and Walter, eds. Immunochemical Methods in Cell and Molecular Biology, Academic Press, London, 1987).

Monoclonal antibodies, reactive against the polypeptide according to the invention (or variants or fragments thereof) according to the present invention, can be prepared by immunising inbred mice by techniques also known in the art (Kohler and Milstein, Nature, 256, 495-497, 1975).

Methods for large-scale production of antibodies according to the invention are also known in the art. Such methods rely on the cloning of (fragments of) the genetic information encoding the protein according to the invention in a filamentous phage for phage display. Such techniques are described i.a at the “Antibody Engineering Page” under “filamentous phage display” at http://aximtl.imt.uni-marburg.de/˜rek/aepphage.html. and in review papers by Cortese, R. et al., (1994) in Trends Biotechn. 12: 262-267., by Clackson, T. & Wells, J. A. (1994) in Trends Biotechn. 12: 173-183, by Marks, J. D. et al., (1992) in J. Biol. Chem. 267: 16007-16010, by Winter, G. et al., (1994) in Annu. Rev. Immunol. 12: 433-455, and by Little, M. et al., (1994) Biotechn. Adv. 12: 539-555. The phages are subsequently used to screen camelid expression libraries expressing camelid heavy chain antibodies. (Muyldermans, S, and Lauwereys, M., Joun. Molec. Recogn. 12: 131-140 (1999) and Ghahroudi, M. A. et al., FEBS Letters 414: 512-526 (1997)). Cells from the library that express the desired antibodies can be replicated and subsequently be used for large scale expression of antibodies.

### EXAMPLES

### Example 1

Isolation of Lawsonia intracellularis from Infected Porcine Ilea.

L. intracellularis infected ilea, confirmed by histopathology and acid-fast Ziebl-Neelsen staining, were collected from pigs died with PE, and stored at −80° C. After thawing L. intracelluris bacteria were isolated from mucosal scrapings taken from the infected intestinal wall. The ileal scrapings were homogenized repeatedly in PBS in an omnimixer to release the intracellular bacteria as described by Lawson et al. (Vet Microbiol. 10: 303-323 (1985)). Supernatant obtained after low-speed centrifugation to remove cell debris was filtered through 5.0, 3.0, 1.2, and 0.8 μm filters (Millipore). The filtrate was subsequently centrifuged at 8000 g for 30 min, giving a small pellet of L. intracellularis bacteria. These bacteria were further purified using a Percoll gradient. The identity of the purified bacteria was assessed by PCR (Jones et al., J. Clin. Microbiol. 31: 2611-2615 (1993)) whereas purity of the isolated bacteria (>95%) was assessed by phase contrast microscopy to reveal any contaminating bacteria or gut debris present.

Bacterial Strains and Plasmids

L. intracellularis cells were isolated from infected ileal material as described above. Escherichia coli host strain BL21star(DE3) containing vector pLysSrare and plasmid pET22b were purchased from Novagen Madison, Wis., USA. E. coli strain TOP10F′ was purchased from Invitrogen (Groningen, the Netherlands). Stocks of all bacterial strains, containing 30% glycerol, were stored at −70° C. Luria Bertani broth (LB) and LB plates were prepared according to standard procedures.

DNA Isolation

In order to obtain highly purified L. intracellularis chromosomal DNA, DNA was prepared from bacterial cells using a Biorad chromosomal DNA isolation kit (Biorad, Veenendaal, the Netherlands). Plasmid DNA was isolated using Qiagen products.

PCR Amplification

PCR amplification was performed using a PCR mixture containing 52 U/ml Expand High Fidelity Enzyme Mix, Expand BF buffer with 2.5 mM MgCl₂, 16 mM dNTPs (Promega, Wis., USA), 20 pmoles of primers and 15 ng chromosomal DNA of L. intracellularis as template.

For standard applications (i.e. colony PCR) the PCR mixture contained 20 U/ml Supertaq and Supertaq buffer (HT Biotechnology Ltd, Cambridge, UK), containing 8 mM dNTPs (Promega, Wis., USA), 10 pmoles of primers and 15 ng template.

Ligation and Transformation

Ligations were performed in a 1× ligation buffer with 1 unit of ligation enzyme (Gibco BRL Life Technologies Inc., USA) at 16° C. overnight 1 μl of the ligation reaction was transformed to E. coli competent cells by heat shock. The BL21star(DE3) E. coli competent cells and the TOP10F′ E coli competent cells were made competent using standard methods.

Expression of 10HIS Fusion Proteins

The DNA sequence of the expression vector was confirmed before the expression vector was transformed to BL21star(DE3) containing pLysSrare. The resulting strain was grown overnight at 37° C. at 200 rpm in 5 ml LB with 100 μg/ml ampicillin. The overnight culture was diluted 1:100 in 50 ml LB with 100 μg/ml ampicillin. This culture was grown under the same conditions until the OD₆₀₀ reached 0.5. The culture was induced with IPTG to a final concentration of 1 mM and continued to grow for a subsequent 3 hours. 100 μl samples were taken for analysis. E. coli strain BL21star(DE3) containing pLysSrare was grown and induced under the same conditions and samples were taken as a negative control. The samples were analyzed by SDS page.

Polyacrylamide Gel Electrophoresis and Western Blotting

SDS-PAGE was performed using 4-2% Bis-Tris gels from the NuPAGE electrophoresis system (Invitrogen, www.invitrogen.com). Western blotting was performed using semi dry blotting procedures. Western blots were developed using chicken anti-Lawsonia polyclonal serum that was raised against a whole cell preparation in a water oil=45:55 emulsion or using a pig serum that had been obtained from a animal that been orally challenged with purified L. intracellularis cells and that had developed clinical signs and post-mortem lesions typical for L. intracellularis infection. The sera were pre-adsorbed using an equal volume crude cell extracts from BL21star(DE3) containing vector pLysSrare at 4° C. for 4 hours.

Results

Cloning of L. intracellaris gene 5608 in T7 Based Expression Vector

Gene 5608 was Amplified using Primer 2179 (CATGCCATGGATTTGATGGAACAGGATTAAAG) and 2180 (CCGCTCGAGCCATAACCCCTTTTTCGATAC). In the process a 5′ NcoI and 3′ XhoI site were introduced into the PCR product. The obtained PCR product was digested using restriction enzymes NcoI and XhoI. The digested PCR product was subsequently ligated to pET22b that had been cut with the same two restriction enzymes. The ligation mixture was transformed to E. coli TOP10F and incubated o/n at 37° C. Putative transformants were checked for the right plasmid, using colony PCR. The plasmid inserts, of colony PCR positive transformants, were checked by nucleotide sequence analysis. One of the clones that contained a sequence as expected on basis of the cloning strategy was chosen and designated pET5608.

Expression of L. intracellularis Gene 5608 from T7 Promoter in E. coli

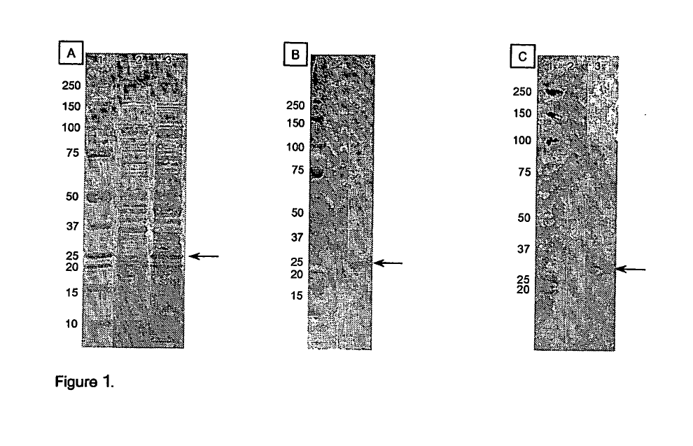

Plasmid pET5608 was transformed to BL21 Star(DE3)pLysSrare. The resulting strain was tested for recombinant protein production as described above. Samples of the induced culture and control samples were analysed by SDS-PAGE gel electrophoresis (FIG. 1A). A clear protein band of approximately 26 kDa was observed in sample that had been taken after 3 hours of induction (FIG. 1A, lane 3) in comparison with the uninduced sample (FIG. 1A, lane 2).

The same samples were also analysed by western blot using the pig and chicken serum A reaction with protein 5608 was observed using the serum from the pig that had been orally challenged with purified L. intracellularis cells (FIG. 1B, lane 3). and with the chicken anti-L. intracellularis serum (FIG. 1C, lane 3).

Conclusion: the 26 kD vaccine component could be successfully expressed in large quantities and is indeed clearly recognised by both orally challenged pig anti-L. intracellularis serum and by chicken anti-L. intracellularis serum.

Legend to the figure.

FIG. 1. Analysis of the overexpression of Lawsonia intracellularis gene 5608 in Escherichia coli BL21 STAR/pLysSRARE by SDS-PAGE (A) and Western blotting with polyclonal pig serum (B) and polyclonal chicken serum (C). Lane 1, molecular weight marker, lane 2, pET5608 T=0; lane 3, pET5608 T=3. Arrows indicate the location of the expression product.

1. A nucleic acid encoding the 26 kD Lawsonia intracellularis protein of claim 6, or a part of said nucleic acid that encodes an immunogenic fragment of said protein.

2. A DNA fragment comprising a nucleic acid according to claim 1.

3. A Recombinant DNA molecule comprising a nucleic acid according to claim 1, under the control of a functionally linked promoter.

4. A live recombinant carrier comprising a nucleic acid according to claim 1.

5. A host cell comprising a nucleic acid according to claim 1.

6. A 26 kD Lawsonia intracellularis protein, said protein comprising an amino acid sequence depicted in SEQ ID NO: 2, or an immunogenic fragment of said protein.

7-8. (canceled)

9. A vaccine for combating Lawsonia intracellularis infection, comprising a nucleic acid according to claim 1, and a pharmaceutically acceptable carrier.

10. The vaccine according to claim 9, comprising an adjuvant.

11. A vaccine according to claim 9, comprising an additional antigen derived from a virus or micro-organism pathogenic to pigs or genetic information encoding said antigen.

12. The vaccine according to claim 11, wherein said virus or micro-organism pathogenic to pigs is selected from the group of Pseudorabies virus, Porcine influenza virus, Porcine parvo virus, Transmissible gastro-enteritis virus, Rotavirus, Escherichia coli, Erysipelothrix rhusiopathiae, Bordetella bronchiseptica, Salmonella cholerasuis, Haemophilus parasuis, Pasteurella multocida, Streptococcus suis, Mycoplasma hyopneumoniae, Brachyspira hyodysenteriae and Actinobacillus pleuropneumoniae.

13. A vaccine for combating Lawsonia intracellularis infections, comprising antibodies against a protein according to claim 6.

14. (canceled)

15. A diagnostic test method for the detection of antibodies against Lawsonia intracellularis, in a specimen, comprising contacting protein or a fragment thereof as defined in claim 6 with the specimen and detecting antibodies bound to the protein.

16. A diagnostic test method for the detection of antigenic material of Lawsonia intracellularis, in a specimen, comprising contacting antibodies against a protein or a fragment thereof as defined in claim 6 with the specimen and detecting antigenic material bound to the antibodies.

17. The nucleic acid of claim 1, comprising the sequence of SEQ ID NO. 1.

18. An isolated protein having an amino acid sequence at least 90% homologous to the protein of claim 6 and having the same antiserum immunoreactivity thereof.

19. A vaccine for combating Lawsonia intracellularis infection, comprising a protein according to claim 6.
